(19) United States
(12) Patent Application Publication
(10) Pub. No.: US 2002/0081676 A1
(43) Pub. Date: Jun. 27, 2002

(54) METHOD FOR PRODUCING AN OXIDE WITH A FERMENTATION PROCESS

(76) Inventors: MASARU YOSHIDA, AICHI (JP); SHINSUKE SOEDA, AICHI (JP); KATUYOSHI HAYASHI, AICHI (JP); HIDEMITSU NANIN, AICHI (JP); YUJI NOGUCHI, AICHI (JP); YOSHIMASA SAITO, HYOGO (JP)

Correspondence Address: OBLON SPIVAK MACCLELLAND, MAIER & NEUSTADT, 1755 JEFFERSON DAVIS HIGHWAY, FOURTH FLOOR, ARLINGTON, VA, 22202

(21) Appl. No.: 09/355,326

(22) PCT Filed: Jan. 26, 1998
(86) PCT No.: PCT/JP98/00301

(30) Foreign Application Priority Data
Jan. 31, 1997 (JP) 9/18248

(51) Int. Cl. C12P 7/50

(52) U.S. Cl. 435/143; 435/41

(57) ABSTRACT

In a method for producing an oxide which comprises cultivating a strain of microorganism of the genus Gluconobacter, the genus Acetobacter, the genus_Pseudogluconobacter, the genus Pseudomonas, the genus_Corynebacterium, or the genus Erwinia to oxidize a substrate in a culture medium, an assimilable carbon source other than the substrate is admixed in the medium.

The above procedure contributes to an increased velocity of oxidation of the substrate in the medium, a reduced fermentation time, an improved fermentation yield, and a reduced percentage of byproducts.

### TECHNICAL FIELD

This invention relates to a method for producing an oxide which comprises cultivating a microorganism selected from the genus Gluconobacter, the genus Acetobacter, the genus Pseudogluconobacter, the genus Pseudomonas, the genus Corynebacterium, or the genus Erwinia to thereby oxidize a substrate in a culture medium.

More particularly, this invention relates to a method for producing an oxide which comprises growing a strain of microorganism of the genus Gluconobacter, the genus Acetobacter, the genus Pseudogluconobacter, the genus Pseudomonas, the genus Corynebacterium, or the genus Erwinia to oxidize a substrate in a culture medium, characterized in that an assimilable carbon source, e.g. a polyhydric alcohol such as a sugar, a sugar alcohol, or glycerol, is admixed in said medium, to a culture medium obtained by practicing the method, and to the oxide obtained by a purification of the said medium.

### BACKGROUND ART

Many strains of microorganisms belonging to the genus Gluconobacter, the genus Acetobacter, the genus Pseudogluconobacter, the genus Pseudomonas, the genus Corynebacterium, or the genus Erwinia have the ability to partially oxidize various substrates such as mono-saccharides, e.g. glucose, fructose, ribose, sorbose, etc., oligosaccharides, e.g. maltose, sucrose, etc., sugar alcohols, e.g. sorbitol, mannitol, ribitol, xylitol, arabitol, etc., or alcohols such as glycerol and ethanol and have been used for the production of useful oxides such as sorbose, 2-keto-L-gulonic acid, acetic acid, and so forth. In connection with this microbiological technology for producing oxides from substrate, much research has been undertaken for improving conversion yields. For this purpose, improvement of microorganisms (Japanese Kokai Tokkyo Koho S62-275692, WO95/23220) and improvement of the cultural method (Japanese Kokai Tokkyo Koho H7-227292), for -instance, have been attempted.

In the hitherto-known processes exploiting a microorganism belonging to the genus Gluconobacter, the genus Acetobacter, the genus Pseudogluconobacter, the genus Pseudomonas, the genus Corynebacterium, or the genus Erwinia for oxidizing a substrate, the conventional mode of addition of a carbon source necessary for growth of the microorganism involves either addition of the substrate alone or addition of a carbon source different from the substrate, together with the substrate, en bloc at initiation of culture. The mode of practice involving addition of the substrate alone has the drawback that the rate of growth of microorganisms is low and this trend is particularly pronounced with strains of microorganisms with a deliberately enhanced efficiency of substrate conversion. Addition of a different carbon source en bloc at initiation of culture for overcoming the above dis-advantage helps to improve the growth rate but results in a decreased specificity of conversion of the substrate compound, not to speak of the problem of increased formation of byproducts. The object of this invention is to provide a technology for increasing the velocity of oxidation of a substrate compound in the medium used for growing a microorganism and thereby reducing the fermentation time, increasing the fermentation yield, and reducing the rate of byproduct formation.

### DISCLOSURE OF INVENTION

After an intensive investigation undertaken in view of the above state of the art, the inventors of this invention found that, in cultivating a microorganism of the genus Gluconobacter, the genus Acetobacter, the genus Pseudogluconobacter, the genus Pseudomonas, the genus Corynebacterium, or the genus Erwinia in a culture medium to oxidize a substrate added to said medium and thereby provide the objective oxide, incorporation of an assimilable carbon source for said microorganism, such as a polyhydric alcohol, e.g. a sugar, a sugar alcohol, or glycerol, in the culture medium in addition to the substrate results in an increased rate of oxidation of the substrate, decreased fermentation time, and increased fermentation yield. This invention has been developed on the basis of the above finding.

This invention, therefore, is directed to a method for producing an oxide which comprises cultivating a microorganism selected from the genus Gluconobacter, the genus Acetobacter, the genus Pseudogluconobacter, the genus Pseudomonas, the genus Corynebacterium, or the genus Erwinia to oxidize a substrate in a culture medium characterized in that an assimilable carbon source is admixed in said medium in the course of the cultivation.

The microorganism of the genus Gluconobacter, the genus Acetobacter, the genus Pseudogluconobacter, the genus Pseudomonas, the genus Corynebacterium, or the genus Erwinia, which is employed in accordance with this invention, can be any strain of microorganism that has the ability to oxidize a substrate compound to provide the objective oxide but is preferably a strain of microorganism with a high conversion efficiency in regard of the oxidation of the substrate to the objective oxide. As such microorganisms with high conversion efficiency, strains known as high-producers of a relevant converting enzyme system, strains elaborating an enzyme system having a high conversion efficiency, strains deficient in the activity to decompose the objective oxides, and strains with an attenuated ability to assimilate the substrate as the sole source of carbon can be mentioned. By way of illustration, when sorbitol is used as the substrate for producing sorbose or 2-keto-L-gulonic acid as the objective oxide or when sorbose is used as the substrate for producing 2-keto-L-gulonic acid as the objective oxide, microorganisms of the genus Gluconobacter or the genus Pseudogluconobacter are preferably used with advantage. Particularly preferred are microorganisms belonging to the genus Gluconobacter. As the examples of such strains of microorganisms, there can be mentioned Gluconobacter oxydans GA-1 (FERM BP-4522), Gluconobacter oxydans N952 (FERM BP-4580) (for both, refer to WO95/23220), Gluconobacter oxydans GO-10 (FERM BP-1169, Gluconobacter oxydans G014 (FERM BP-1170) (for both refer to Japanese Kokai Tokkyo Koho S62-275692), Gluconobacter oxydans UV-10 (FERM P-8422), Gluconobacter oxydans E-1 (FERM P-8353), all of which belong to the species of Gluconobacter oxydans, and Pseudogluconobacter K591s (FERM BP-1130), Pseudogluconobacter 12-5 (FERM BP-1129), Pseudoglucono-bacter TH14-86 (FERM BP-1128), Pseudogluconobacter 12-15 (FERM BP-1132), Pseudogluconobacter 12-4 (FERM BP-1131), and Pseudogluconobacter 22-3 (FERM BP-1133), all of which belong to the genus Pseudogluconobacter.

The culture method for use in the practice of this invention can be appropriately selected according to the strain of microorganism, the substrate compound, and the objective compound, among other factors, and a known cultural procedure such as shake culture or submerged aerobic culture can be employed.

The substrate that can be used in the method of this invention includes monosaccharides such as glucose, fructose, ribose, sorbose, etc., oligosaccharides such as maltose, sucrose, etc., sugar alcohols such as sorbitol, mannitol, ribitol, xylitol, arabitol, etc., and alcohols such as glycerol and ethanol. The amount of addition of the substrate varies with the kind of strains of micro-organisms, cultural procedures, and species of substrate but is generally 1 to 50%, preferably 3-20%, of the culture medium.

There is no particular limitation on the kind of assimilable carbon source other than said substrate as far as microorganism is able to assimilate it. When, for instance, the strain of microorganism is one having the ability to act upon sorbitol or sorbose to produce sorbose or 2-keto-L-gulonic acid, said carbon source can be selected from among sugars (e.g. oligosaccharises such as sucrose, maltose, etc. and monosaccharides such as glucose, fructose, etc.), sugar alcohols (e.g. sorbitol, mannitol, xylitol, etc.), and polyhydric alcohols such as glycerol. Among such polyhydric alcohols, glycerol is particularly preferred because it contributes a great deal to improvements in the efficiency and velocity of conversion and a reduced amount of products of incomplete metabolism.

The amount of said carbon source varies with the kind of strains of microorganisms, cultural procedures, carbon sources, substrate compounds, and amounts of the substrate compound but may range from 1 to 100%, preferably from 10 to 50%, of the amount of the substrate.

The mode of addition of said carbon source varies with the kind of strains of microorganisms, cultural procedures, carbon sources and substrates but it can be added in the course of the cultivation. More specifically, the period of addition of said carbon source can be selected a certain time after initiation of culture, either continuously or at intervals, and in predetermined portions, or according to the progress of fermentation.

This invention can be effectively carried out by adding natural organic nutrients such as yeast extract, dried yeast, corn steep liquor, etc. as auxiliary nutrients in addition to said substrate and carbon source in order to accelerate growth of the microorganisms and maintain a sufficient conversion activity.

The objective oxide produced by working this invention can be harvested and purified by known means to the ordinally skilled in the art according to the kind of oxide. It may also be isolated in the form of a salt, such as the sodium salt or the calcium salt. Isolation can, for example, be made by subjecting the culture medium to filtration or centrifugation, with or without active carbon treatment, for removing the cells and, then, subjecting the liquid fraction to crystallization by concentration, adsorption on a resin, chromatography, salting-out, etc. as applied singly, in a suitable combination, or in repetition.

This invention provides an economical and efficient technology for the industrial production of an oxide which comprises cultivating a microorganism belonging to the genus Gluconobacter, the genus Acetobacter, the genus Pseudogluconobacter, the genus Pseudomonas, the genus Corynebacterium, or the genus Erwinia in a culture medium for oxidizing a substrate in the medium, which provides for an accelerated oxidation rate, reduced fermentation time, and improved fermentation yield.

### EXAMPLE 1

A culture medium (50 ml) containing 0.5% glucose, 5% sorbitol, 1.5% corn steep liquor, and 0.15% magnesium sulfate in a 500 ml flask was inoculated with 0.5 ml of a liquid nitrogen-preserved culture of Gluconobacter oxydans N952 (FERM BP-4580), a transformant of Gluconobacter oxydans (WO95/23220), and incubated at 30° C. for 24 hours. A portion (17 ml) of this culture was transferred to a 30-L jar fermenter containing a sterilized medium (17 L) of the same composition as above and incubated at 30° C. for 20 hours. A 2 L portion of this seed culture was transferred to a 30 L jar fermenter containing a culture medium (17 L) containing 15% sorbitol, 2% corn steep liquor, 0.3% yeast extract, 0.5% magnesium'sulfate, and 0.5% calcium carbonate and incubated at 32° C. for 70 hours. In the course of this culture, the medium was controlled at pH 5.5 up to 24 hours and, then, at pH 6.5 till completion of fermentation by adding an aqueous solution of sodium hydroxide and agitated by sparging to maintain dissolved oxygen at 10% or higher. The culture broth thus obtained was used as control. On the other hand, the same strain of microorganism was cultured with continuously addition of glycerol in an amount corresponding to 6% of the final culture medium from the initiation 13.5 hours after the initiation of culture till completion of fermentation (after 70 hours from the initiation of cultivation) under otherwise the same conditions. The efficiency of conversion from sorbitol to 2-keto-L-gulonic acid was 41.3% in the experiment involving addition of glycerol, demonstrating a remarkable effect as compared with the control experiment without addition of glycerol (24.8%) at the 70 hours from the initiation of culture.

### EXAMPLE 2

Using Gluconobacter oxydans HS17 [Gluconobacter_oxydans NB6939-pSDH-tufB1 (WO95/23220) subjected to nitrosoguanidine-induced mutagenesis for enhancing the efficiency of conversion from sorbitol to 2-keto-L-gulonic in lieu of Gluconobacter oxydans N952, the cultural procedure of Example 1 was otherwise repeated. Addition of glycerol began from 13 hours from the initiation of culture till 72 hours from the initiation of culture till 72 hours in an amount corresponding to 6% of the final culture medium. In a control experiment, glycerol was added en bloc in an amount corresponding to 6% of the final culture medium before the initiation of the culture. The efficiencies of conversion from sorbitol to 2-keto-L-gulonic acid were measured and compared between experiments at 24, 48, 56 and 72 hours after the initiation of culture and the control medium respectively. The results are shown in Table 1.

TABLE 1

| | After | After | After | After |
|---|---|---|---|---|
| | 24 hr | 48 hr | 56 hr | 72 hr |
| Addition en bloc | 22% | 42% | 45% | ND* |
| Before cultivation | | | | |
| Addition begun | 25% | 74% | 85% | 90% |
| From at 13 hr till | | | | |
| 24, 48, 56 or 72 hrs. | | | | |

*ND: not measured

1. A method for producing an oxide which comprises cultivating a microorganism selected from the genus Gluconobacter, the genus Acetobacter, the genus Pseudogluconobacter, the genus Pseudomonas, the genus Corynebacterium, or the genus Erwinia to oxidize a substrate in a culture medium characterized in that an assimilable carbon source is admixed in said medium.

2. A method for producing an oxide according to claim 1 wherein the assimilable carbon source is a polyhydric alcohol.

3. A method for producing an oxide according to claim 1 wherein the assimilable carbon source is a member selected from the group consisting of glycerol, monosaccharides and sugar alcohols.

4. A method for producing an oxide according to claim 1 wherein the assimilable carbon source is glycerol.

5. A method for producing an oxide according to claims 1 through 4 wherein the substrate in the culture medium is sorbitol or sorbose.

6. A method for producing an oxide according to claims 1 through 5 wherein the oxide is 2-keto-L-gulonic acid.

7. A method for producing an oxide according to claims 1 through 6 wherein the microorganism is Gluconobacter oxydans.

8. A culture medium obtained by the method claimed in claims 1 through 7.

9. The oxide obtained by a purification of the culture medium of claim 8.
